(19) United States
(12) Patent Application Publication
(10) Pub. No.: US 2024/0156381 A1
(43) Pub. Date: May 16, 2024

(54) INFORMATION PROCESSING APPARATUS, INFORMATION PROCESSING METHOD, AND PROGRAM

(71) Applicant: SONY GROUP CORPORATION, TOKYO (JP)

(72) Inventors: OSAMU SHIGETA, TOKYO (JP); SHIN SHIROMA, TOKYO (JP); ITARU SHIMIZU, TOKYO (JP); TETSUO SAKAMOTO, TOKYO (JP); TOMOMITSU HERAI, TOKYO (JP)

(21) Appl. No.: 18/551,034

(22) PCT Filed: Jan. 25, 2022
(86) PCT No.: PCT/JP2022/002505

§ 371 Date: Sep. 18, 2023

(30) Foreign Application Priority Data
Mar. 31, 2021 (JP) 2021-059262

(51) Int. Cl. A61B 5/16; A61B 5/024; A61B 5/0531

(52) U.S. Cl. CPC A61B 5/16; A61B 5/024; A61B 5/0531

(57) ABSTRACT

Provided is an information processing apparatus including an estimation part configured to estimate a load experienced by a user, on the basis of data from a sensor measuring a physiological index of the user, the estimation part estimating, as the load, at least one of a workload experienced by the user carrying out a predetermined task and a visual load experienced by the user making alert concentration.

### TECHNICAL FIELD

The present technology relates to an information processing apparatus, an information processing method, and a program. For example, this technology relates to an information processing apparatus, an information processing method, and a program for estimating a stress experienced by a user and performing processes to reduce the stress.

### BACKGROUND ART

There has been proposed a stress measurement method for estimating a stress level of a user by analyzing the heart rate of the user (see PTL 1). There have also been proposed methods for reducing a stress according to its level.

### CITATION LIST

### Patent Literature

### [PTL 1]

Japanese Patent Laid-open No. 2012-120206

### SUMMARY

### Technical Problem

However, the above-mentioned proposals focus only on the stress level and fail to consider factors causing the stress. There has thus been a need for a scheme of reducing the stress of the user by carrying out suitable processes reflecting the cause of the stress.

The present technology has been devised in view of the above circumstances and aims to execute appropriate processes to reduce stress according to the cause thereof.

### Solution to Problem

According to one aspect of the present technology, there is provided an information processing apparatus including an estimation part configured to estimate a load experienced by a user, on the basis of data from a sensor measuring a physiological index of the user. The estimation part estimates, as the load, at least one of a workload experienced by the user carrying out a predetermined task and a visual load experienced by the user making alert concentration.

According to another aspect of the present technology, there is provided an information processing method including causing an information processing apparatus to estimate a load experienced by a user on the basis of data from a sensor measuring a physiological index of the user, and causing the information processing apparatus to estimate, as the load, at least one of a workload experienced by the user carrying out a predetermined task and a visual load experienced by the user making alert concentration.

According to a further aspect of the present technology, there is provided a program for causing a computer controlling an information processing apparatus to execute a process including estimating a load experienced by a user on the basis of data from a sensor measuring a physiological index of the user, and estimating, as the load, at least one of a workload experienced by the user carrying out a predetermined task and a visual load experienced by the user making alert concentration.

According to the above-mentioned aspects of the present technology, there are thus provided the information processing apparatus, the information processing method, and the program for estimating the load experienced by the user on the basis of the data from the sensor measuring the physiological index of the user. What is estimated as the load is at least one of a workload experienced by the user carrying out a predetermined task and a visual load experienced by the user making alert concentration.

Incidentally, the information processing apparatus may be either an independent apparatus or an internal block constituting part of a single apparatus.

Also, the program can be transmitted via a transmission medium or recorded on a recording medium when offered.

### BRIEF DESCRIPTION OF DRAWINGS

FIG. 1 is a table explaining visual loads and workloads.

FIG. 2 is a diagram explaining relations between physiological indexes and loads.

FIG. 3 is a diagram explaining RRI.

FIG. 4 is a diagram explaining analysis intervals.

FIG. 5 is a diagram depicting an exemplary configuration of an information processing apparatus.

FIG. 6 is a diagram depicting an exemplary configuration of another information processing apparatus.

FIG. 7 is a flowchart explaining a process related to interventions.

FIG. 8 is a flowchart explaining another process related to interventions.

FIG. 9 is a diagram depicting an exemplary configuration of a personal computer.

### DESCRIPTION OF EMBODIMENT

What follows is a description of an embodiment for implementing the present technology (referred to as the embodiment hereunder).

### <Relation Between Workload and Stress>

The present technology may be applied to cases where measurements made regarding a stress experienced by a user are used to help reduce the stress.

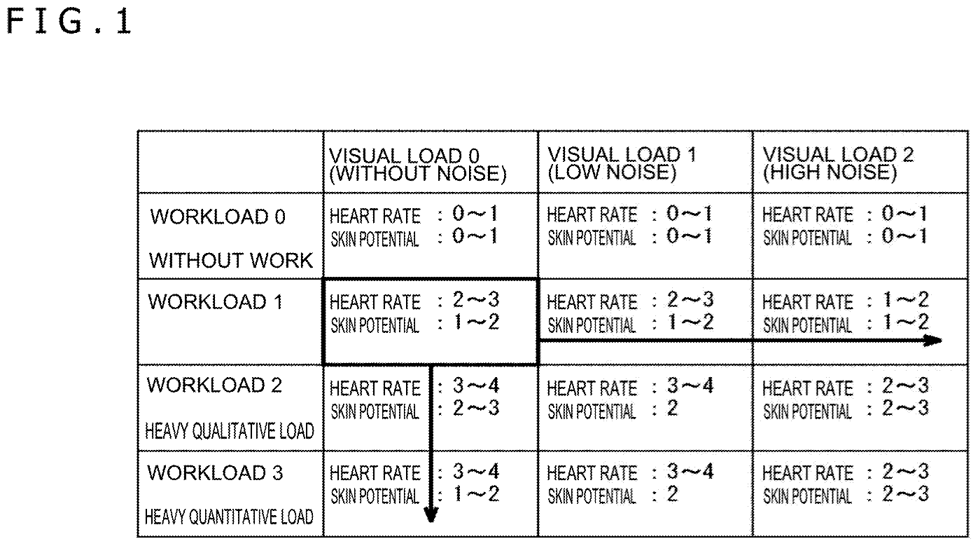

This applicant conducted experiments in which relations between the load experienced by a user carrying out a predetermined task and the stress experienced by the user carrying out the task are examined. The results of the experiments are indicated in FIG. 1.

FIG. 1 is a table indicating results of measuring changes in the measurements of the heart rate and skin potential of the user (test subject) subjected to workloads and visual loads.

The workload is a load experienced by the user carrying out a specific task. In particular, the workload refers to a cognitive load resulting from executing the task and a load stemming from consuming attention resources during task execution. The workload may also be regarded as a fatigue or stress accumulated by the user carrying out a specific task.

For example, in a case where a user remotely controls a drone, the user (operator in this case) experiences a load stemming from the task of operating the drone. Also, during remote control of the drone, the user controls the movement of the drone while watching images coming from the drone. This means that there is a load resulting from recognizing the state of the drone by watching the images as well as a load stemming from consuming visual resources by watching the images and from consuming resources necessary for operating a remote controller of the drone.

Also, the workloads fall into two categories: a load related to work content and a load related to work volume. The load related to work content (referred to as the qualitative load hereunder) is a load experienced by the user carrying out a highly difficult task or a task requiring precise operations. The qualitative load is a load experienced by the user carrying out work (tasks) per unit time.

The load related to work volume (referred to as the quantitative load hereunder) is a load experienced by the user having to deal with many tasks or performing time-consuming work. The qualitative load is a workload accumulated by continuously performing work (tasks).

Given the same task, different users experience different load amounts. If the same quantitative load is given to different users, for example, there are individual differences between the different users experiencing different load amounts. Some users skilled in a given task are expected to experience a lower load amount carrying out the task while novices are likely to experience a higher load amount under the same task. To absorb the individual differences between the different users experiencing different loads carrying out the same task, the experimental results in FIG. 1 indicate normalized results from multiple test subjects.

Exemplary tasks generating the workload are the tasks of monitoring work. Such tasks include a task requiring continuous attention to a monitoring target and a task of checking the monitoring target. Other exemplary tasks causing workloads are tasks during game play, including game operations such as the manipulation of characters during games and the input of commands.

Also, other exemplary tasks generating the workload are tasks of driving a mobility device. The mobility devices are mobile objects such as aircraft, electric trains, and vehicles. Drones and robots may also be included in the mobility devices. The tasks of driving the mobility device include all tasks required to reach the destination by operating the mobility device, including a task of operating the mobility device, a task of monitoring the state of the mobility device, and a task of monitoring the surroundings.

The experimental results in FIG. 1 are the workloads measured when some of the above tasks were actually carried out or the workloads measured when pseudo tasks simulating those tasks were executed.

In the table in FIG. 1, the items referred to as workload 0, workload 1, workload 2, and workload 3 are listed in the vertical direction. The item called workload 0 describes results obtained in a case where no work was performed and the test subject was at rest.

The item called workload 1 describes results obtained in a case where a predetermined task was carried out. The predetermined task is a task that serves as the reference with respect to workload 2 and workload 3, and thus may be referred to as the reference task hereunder where appropriate. The item called workload 1 describes results obtained in a case where the user performed the reference task.

The item called workload 2 describes results obtained in a case where a task with a heavy qualitative load was performed. The task with the heavy qualitative load is a task of which the qualitative load is heavier than that of the reference task. The tasks with heavy qualitative loads include a task involving a large amount of necessary work, a task with its results affecting the user significantly, and a task involving complicated operations.

Also, the task with the workload 2 in the case where the experimental results in FIG. 1 were obtained is a task involving approximately 1.5 to 2.0 times the work per unit time or the psychological load of the reference task.

The workload 3 describes results obtained in a case where a task with a heavy quantitative load was carried out. The task with the heavy quantitative load is a task of which the quantitative load is heavier than that of the reference task. The tasks with heavy quantitative loads are tasks involving work carried out for an extended period of time.

The task with the workload 3 in the case where the experimental results in FIG. 1 were obtained is a task involving approximately 1.5 to 2.0 times the work time of the reference task.

The items called visual load 0, visual load 1, and visual load 2 are listed on the horizontal axis in FIG. 1. An explanation is made below regarding the visual load.

The visual load is a load experienced by the user (test subject) making a visual concentration. The visual load is, for example, a load experienced by the user who, while viewing the display, tries to concentrate to obtain visual information despite disturbances to the ongoing visual presentation, such as a disturbed screen (generation of noise), image delays, a disturbed viewpoint position, or a disturbed stereoscopic view.

For example, in the case of visual recognition in real-life settings such as that during driving of a vehicle, a load may be experienced by concentrating to try to obtain visual information despite poor visibility such as the field of view being disturbed by rainfall or fog. Also, during visual recognition in real-life settings, the field of view may be limited by disturbances other than the weather conditions such as the rainfall or fog.

Given the same load factor, the visual load varies in amount from individual to individual. For example, during viewing of the display, a disturbed screen may not be much of a load on those who are used to such a disturbance but may be highly burdensome on those not accustomed to it. The experimental results obtained from multiple test subjects as indicated in FIG. 1 have thus been normalized in order to absorb such individual differences in visual load, as in the case of the above-described qualitative load.

The visual load may also be considered “alert concentration.” Alert concentration may be regarded as making concentration to deal with unpredictable states or unexpected situations. For example, during driving of the vehicle, it is necessary to concentrate in a manner paying extensive attention to the surroundings so as to handle the unpredictable events such as rush-out of children or a vehicle coming from an unexpected direction. It is also necessary to drive while remaining alert to such unpredictable situations taking place at any moment.

Since such alert concentration is acquired primarily from visual information, the concentration constitutes a load experienced by the user as a visual load. Whereas the description that follows uses the visual load as an example, the present technology also covers cases of keeping alert concentration on the information obtained from senses other than sight, such as hearing and touch. For example, in a case where caution is exercised to look out for rush-out of anything unexpected, the user may concentrate to obtain more auditory information. In such a case, the concentration is also experienced by the user as a load. Such auditory load may thus be handled in a manner similar to the visual load.

Some exemplary tasks generating the visual load are those in monitoring work. For example, there are tasks which, in the case of remote monitoring during viewing of the display, require increased concentration due to visual disturbances such as noise or delays in the image on the display. There are other tasks that require making alert concentration on the effects of weather conditions or on other sudden disturbances during remote monitoring.

Other exemplary tasks generating the visual load are those during game play. For example, there are tasks that incur situations requiring increased concentration due to visual disturbances such as noise or delays on the screen during game play. There are other tasks that require making alert concentration on disturbances that impede games, including the occurrence of a disrupting event during game play or a possible intervention with regard to the user playing the game.

Other exemplary tasks generating the visual load are those during driving of a mobility device. For example, there are tasks that incur situations requiring increased concentration on driving of the mobility device due to visual disturbances such as the effects of weather conditions. There are other tasks that also require increased concentration on alert for disturbances that disrupt the driving of the mobility device, including interventions by passers-by.

The experimental results in FIG. 1 are those measured in the case where some of these tasks were actually carried out or in the case where pseudo tasks simulating those tasks were executed, as the visual load.

In the table in FIG. 1, the item called visual load 0 placed in the horizontal direction describes results obtained when work was performed during viewing of a noise-free screen.

The item called visual load 1 describes results obtained in a case where work was performed during viewing of a low-noise image. The item called visual load 2 describes results obtained in a case where work was carried out during viewing of a high-noise image.

With reference to FIG. 1, when the user was made to perform work with workload 0 under visual load 0, visual load 1, and visual load 2, the result was that the heart rate was 0 to 1, and the skin potential was 0 to 1. That is, in the case of the workload 0 or during rest, the heart rate and the skin potential remained stable regardless of the visual load.

The values such as 0 to 1 indicative of the heart rate and skin potential are normalized values, which represent a 10-stage evaluation of the heart rate and skin potential. In this case, it is assumed that these are evaluated values representing the amounts of change on a scale of 10 starting from a resting state.

The heart rate is the number of times the heart pulsates in a predetermined period of time. Generally, the heart rate is represented by the number of times the heart pulsates per minute (BPM: Beat Per Minutes). The values such as 0 to 1 of heart rate in FIG. 1 are normalized values providing easy-to-understand comparisons of the heart rate corresponding to the workloads and visual loads with respect to the heart rate at rest taken as the reference, for example.

As examples of normalization, in a case where the differential value with respect to the reference heart rate is 0 to 10, the heart rate may be expressed as heart rate 0; in a case where the differential value is 10 to 20, the heart rate may be expressed as heart rate 1; and in a case where the differential value is 20 to 30, the heart rate may be expressed as heart rate 2.

In a case where the heart rate is close to the reference value, the change in heart rate resulting from the change in load is expected to increase. The higher the heart rate, the smaller the change in heart rate resulting from the change in load is expected. The range of the differential value may be extended in a case where the heart rate is close to the reference value, and the range of the differential value may be reduced in a case where the heart rate is far from the reference value. In normalization, in a case where the differential value with respect to the reference value is 0 to 10, the heart rate may be expressed as heart rate 0; in a case where the differential value is 10 to 18, the heart rate may be expressed as heart rate 1; and in a case where the differential value is 18 to 24, the heart rate may be expressed heart rate 2, for example.

It is to be noted that heart rate 2 is not in double relation with heart rate 1. What is expressed here is a measure of magnitude relation in tendency, and heart rate 2 is simply larger than heart rate 1.

The normalization of skin potential (skin conductance) is basically similar to that of heart rate. Skin potential is measured in units of μS (micro-siemens). The values of skin potential are also normalized values providing easy-to-understand comparisons of the skin potential corresponding to the workloads and visual loads with respect to the skin potential at rest taken as the reference, for example.

For example, it is also to be noted that skin potential 2 is not in double relation with skin potential 1. What is indicated is a measure of magnitude relation in tendency, and skin potential 2 is simply larger than skin potential 1.

In a case where the user was made to perform work with workload 1 under visual load 0, the heart rate was 2 to 3, and the skin potential was 1 to 2. Also, in a case where here the user was made to carry out work with workload 1 under visual load 1, the heart rate was 2 to 3, and the skin potential was 1 to 2. In a case where the user was made to conduct work with workload 1 under visual load 2, the heart rate was 1 to 2, and the skin potential was 1 to 2.

The case where the user was made to perform work with workload 1 with the visual load allowed to change is a state under visual load 2, i.e., a high-noise state, in which the screen was viewed more attentively. It can be seen that, with alert concentration, the heart rate dropped but the skin potential remained unchanged.

In a case where the user was made to perform work with workload 2 under visual load 0, the heart rate was 3 to 4, and the skin potential was 2 to 3. In a case where the user was made to carry out work with workload 2 under visual load 1, the heart rate was 3 to 4, and the skin potential was 2. In a case where the user was made to conduct work with workload 2 under visual load 2, the heart rate was 2 to 3, and the skin potential was 2 to 3.

It can be seen that, also in a case where the user was made to perform work with workload 2 with the visual load allowed to change, as in the case where the user was made to carry out work with workload 1 with the visual load allowed to change, a heavier visual load led to a lower heart rate, with the skin potential staying unchanged.

In a case where the user was made to perform work with workload 3 under visual load 0, the heart rate was 3 to 4, and the skin potential was 1 to 2. In a case where the user was made to carry out work with workload 3 under visual load 1, the heart rate was 3 to 4, and the skin potential was 2. In a case where the user was made to conduct work with workload 3 under visual load 2, the heart rate was 2 to 3, and the skin potential was 2 to 3.

In a case where the user was made to perform work with workload 3 with the visual load allowed to change, as in the case where the user was made to carry out work with workload 1 with the visual load allowed to change, a heavier visual load led to a lower heart rate. In this case, it can be seen that the heavy visual load led to a higher skin potential.

Next, the changes in heart rate and skin potential are read from the results found in FIG. 1 in the vertical direction.

In a case where the user was made to perform work with workload 1 under visual load 0, the heart rate was 2 to 3, and the skin potential was 1 to 2. In a case where the user was made to carry out work with workload 2 under visual load 0, the heart rate was 3 to 4, and the skin potential was 2 to 3. In a case where the user was made to conduct work with workload 3 under visual load 0, the heart rate was 3 to 4, and the skin potential was 1 to 2.

It can be seen that, in the case where the user was made to work under visual load 0 with the workload allowed to change, the heart rate was high under workload 2, i.e., when the qualitative load was heavy, and was also high under workload 3, i.e., when the quantitative load was heavy. It can also be seen that the skin potential was high when the qualitative load was heavy and remained unchanged when the quantitative load was heavy.

In a case where the user was made to perform work with workload 1 under visual load 1, the heart rate was 2 to 3, and the skin potential was 1 to 2. In a case where the user was made to carry out work with workload 2 under visual load 1, the heart rate was 3 to 4, and the skin potential was 2. In a case where the user was made to conduct work with workload 3 under visual load 1, the heart rate was 3 to 4, and the skin potential was 2.

It can be seen that, in the case where the user was made to work under visual load 1 with the workload allowed to change, the heart rate was high under workload 2, i.e., when the qualitative load was heavy, and was also high under workload 3, i.e., when the quantitative load was heavy. It can also be seen that the skin potential remained unchanged both when the qualitative load was heavy and when the quantitative load was heavy.

In a case where the user was made to perform work with workload 1 under visual load 2, the heart rate was 1 to 2, and the skin potential was 1 to 2. In a case where the user was made to carry out work with workload 2 under visual load 2, the heart rate was 2 to 3, and the skin potential was 2 to 3. In a case where the user was made to conduct work with workload 3 under visual load 2, the heart rate was 2 to 3, and the skin potential was 2 to 3.

It can be seen that, in the case where the user was made to work under visual load 2 with the workload allowed to change, the heart rate was high under workload 2, i.e., when the qualitative load was heavy, and was also high under workload 3, i.e., when the quantitative load was heavy. It can also be seen that the skin potential was high when the qualitative load was heavy and was also high when the quantitative load was heavy.

From the experimental results in FIG. 1, it can be seen that, when the visual load was heavy, the skin potential remained unchanged but the heart rate tended to drop. It can also be seen that, when the qualitative load was heavy as the workload, the heart rate and the skin potential tended to increase. It can further be seen that, when the quantitative load was heavy as the workload, both the skin potential and the heart rate tended to increase.

An explanation is further made below regarding the changes in heart rate and skin potential occurring when workloads and visual loads were raised and lowered, as found from the experimental results in FIG. 1.

As its characteristic, the heart rate is known to vary depending on the balance between the sympathetic and parasympathetic nervous systems. For this reason, changes in the balance between the sympathetic and parasympathetic nervous systems are presumably estimated by measuring the heart rate. However, consideration should also be given to cases where the heart rate is changed by factors other than the balance between the sympathetic and parasympathetic nervous systems and where the sympathetic and parasympathetic nervous systems are affected by other events that may lead to changes in heart rate.

The heart rate may also be affected and changed by both mental activity and body response. For example, the heart rate is known to vary due to diverse factors such as temperature change, body movement, breathing, psychological anxiety, and mental stress. Given this knowledge, measuring the heart rate presumably makes it possible to estimate whether the test subject is in a state of being affected by mental activity or body response.

From the experimental results in FIG. 1, it can be found that the heart rate is low in the case where the visual load is heavy. One reason the heart rate is low when the visual load is heavy is that the heart rate is reduced probably because of concentration. This mechanism presumably involves the possibility of the parasympathetic nervous system being activated.

Under heavy load conditions, the heart rate is expected to increase in the case of both heavy quantitative load and heavy qualitative load. Still, the parasympathetic nervous system may not be the only factor for the high heart rate. Other factors might possibly contribute to it.

Such other factors may include two types of concentration: absorbed concentration and alert concentration. In the case of alert concentration, the heart rate is reduced presumably for risk aversion. Absorbed concentration is a state of such deep absorption in reading, for example, that the reader is unaware of the surroundings. This is a state of intense absorption in something and of focused concentration thereon.

Alert concentration is a state of being on alert for unexpected events, i.e., a state of concentration on averting such events. An example of alert concentration is that, during driving of the vehicle, the driver is on alert for rush-out of persons and is paying extensive attention to the surroundings.

Skin potential has the characteristic of responding to the activity of the sympathetic nervous system. The factors for activating the sympathetic nervous system are known to be of two kinds: factors attributable to mental stress, and other factors. Those other factors include the case of a close call. A close call is a case of acknowledging an event that may well have led to, but just stopped short of, a disaster or a serious accident. On the basis of this, measuring the skin potential presumably makes it possible to estimate whether the person being measured is in a state of mental stress or a close call affecting the activity of the sympathetic nervous system.

From the experimental results in FIG. 1, it can be found that the skin potential is not affected by the visual load. Since the skin potential responds to the activity of the sympathetic nervous system as discussed above, the sympathetic nervous system is found not affected by the visual load that does not affect the skin potential.

The skin potential is expected to increase in a case where the sympathetic nervous system is activated by an increased qualitative load of the task. Also, in a case where the quantitative load of the task is raised, the skin potential is expected to be not much affected thereby.

Other factors regarding the skin potential include a case where the skin potential is brought into a further active state due to a surprise upon start of a task, for example.

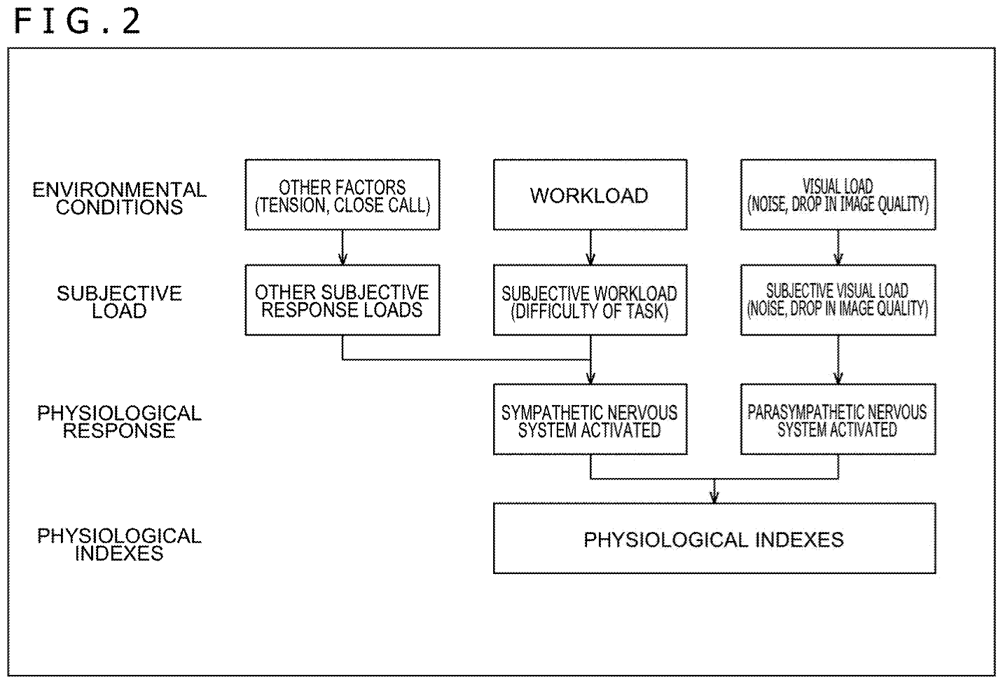

Through the use of what was discussed above, how to measure whether or not the user feels tired or stressed when carrying out a predetermined task is explained below with reference to FIG. 2.

In a case where the environmental conditions are changed such as where a noise has occurred in the image being displayed or where resolution has dropped leading to reduced image quality, a visual load is generated. The visual load, when generated, causes the user subjectively to experience a load such as stress or fatigue (subjective visual load). Under subjective visual load, the parasympathetic nervous system of the user is activated. As a result, the physiological indexes of the user such as the heart rate are changed.

In a case where the environmental conditions are changed such as where working time is prolonged or where the level of difficulty of work content is raised, a workload is generated. The workload, when generated, causes the user subjectively to experience a load such as stress or fatigue (subjective workload). Under subjective workload, the sympathetic nervous system of the user is activated. As a result, the physiological indexes of the user such as the heart rate and the skin potential are changed.

In a case where the environmental conditions are changed due to factors other than those regarding the above-mentioned visual load and workload, such as where the situation has incurred high tension or where the circumstances require being on alert for a close call, the user experiences a different subjective reaction load. Under such different subjective reaction load, the sympathetic nervous system of the user is activated. As a result, the physiological indexes of the user such as the heart rate and the skin potential are changed.

In such a manner, in the case where the environmental conditions are changed (where the workload or visual load is changed), a physiological response occurs, which causes the physiological indexes to change. Through the use of these findings, measuring the changes in the physiological indexes makes it possible to detect changes in the environmental conditions (workload and visual load).

For example, in a case where the physiological indexes such as the heart rate and the skin potential are changed, it can be estimated that the user is experiencing a load and that the loaded state may have been brought about by changes in the environmental conditions.

There are cases where the changes in the environmental conditions are previously known to the system side. For example, in a case where the user is playing a game, the apparatus side offering the game is cognizant of the content of the tasks offered to the user, such as what operations are required of the user and how high the level of difficulty of game content is.

In exemplary cases where the environmental conditions are previously known and where it can be determined that the user is experiencing a certain load on the basis of the physiological indexes, the apparatus side offering the tasks may make arrangements to perform processes for changing the environmental conditions so as to reduce the load. For example, in a case where the above-mentioned user is playing the game and the user is found to be experiencing a load based on the physiological indexes, suitable processes may be carried out to lower the level of difficulty of game content or to change the color or resolution of the screen for easier game viewing.

### <Examples of Estimation and Intervention>

Explained below with specific examples are what can be measured as the physiological indexes, what can be estimated from such measurements, and how to intervene to reduce the load experienced by the user on the basis of the estimated results.

As case 1, the amount of activity of the parasympathetic nervous system can be measured by keeping track of the physiological indexes. Measuring the amount of activity of the parasympathetic nervous system makes it possible to estimate whether or not the user is in a state of alert. In a case where it is determined that the user is in a state of alert, an intervention is made to reduce the degree of alert (i.e., to reduce the load experienced by the user).

For example, in a case where the physiological indexes of the driver in semi-autonomous driving of a mobility device are measured and the driver is thereby determined to be in a state of alert, the mobility device is controlled in a manner supporting the driver. An intervention is thus made to reduce the load of and the disturbances to the driver. For example, in a case where it has started to rain, which has worsened visibility, and the poor visibility is found to put a load on the user, the wipers of the mobility device may be suitably controlled without bothering the driver. Also in a case where the load on the user is found to be heavy, announcements to the user may be controlled to be suspended or to be made after the load on the user is reduced.

Also, an intervention to reduce the load on the user may be made by reinforcing assist functions such as adjustment of the inter-vehicle distance (adjustment of speed). Also, an intervention to reduce the load on the user may also be made by displaying navigation instructions at a position easily viewed by the user through AR (Augmented Reality).

Also, an intervention may also be made to relax the driver of the mobility device or the game player in an alert state. Interventions of this type may include presenting a voice message or a piece of music encouraging the person to take a rest.

Conversely, in a case where the driver or the game player is not on alert when they should be, an intervention may be made to prompt the driver to be on alert or to make things harder for the game player by increasing the number of enemy characters, for example.

As case 2, the degree of subjective workload is estimated by measuring the quality of the image presented by the system (amount of noise and resolution) and by determining whether the sympathetic nervous system is dominant. If it is determined that there is a heavy subjective workload, an intervention is made to reduce the workload. An intervention is also made to introduce tasks with light workloads. Interventions of this type include cases where there is a state of concentration but the workload is light.

As case 3, the amount of activity of the sympathetic nervous system and that of the parasympathetic nervous system are measured. In a case where the quality of communication is previously known to the system side, whether the current load is due to a workload or a visual load can be estimated. If it is determined that there is a heavy visual load, additional processes are performed to secure image quality as much as possible.

For example, in a case where the device such as a drone or a robot is remotely operated and such operations are carried out by viewing images on the display, there is a possibility that the images captured by the device are delayed before being displayed. If communication delays occur, delayed operations performed on the drone may not be quick enough to avoid obstacles. In the case where there are communication delays, delay-free communication may be established by narrowing the bandwidth of the communication network in use to reduce image quality.

When image equality drops, there is a possibility that the visual load increases and so does the user's subjective visual load. In such a case, processes are carried out to secure image quality as much as possible. For example, an intervention may be made to switch to the task content tolerating communication delays or to widen the bandwidth of communication in order to improve image quality. An intervention may also be made to increase data redundancy.

In a case where the status of load on the user is monitored and the load is found to be reduced, processes may be performed to eliminate communication delays rather than improve image quality. In a case where the load is found to become heavier still, processes may be carried out to ensure communication with higher image quality. In this manner, available resources may be apportioned in handling image quality depending on the result of the monitoring.

On the other hand, in a case where the visual load is determined to be light, the image quality and communication delays at that point in time may be maintained. In other words, in a case where the user is not experiencing a load despite long delays, low bandwidth, or low image quality, no intervention may be needed.

### <Measurement of Physiological Indexes>

The load experienced by the user is estimated by measuring the physiological indexes such as the user's heart rate and skin potential. An additional explanation is made below of how to measure the physiological indexes.

In a case where the heart rate is measured as a physiological index, a heart rate meter may be used. The heart rate meter may be a wearable device such as a smartwatch attached to the user's body or may be included in a VR (Virtual Reality) headset worn by the user.

The heart rate may also be measured by capturing images of the user and analyzing the captured images. Specifically, the heart rate may be measured by estimation using a scheme of measuring the hemoglobin level in the blood. As another alternative, the heart rate may be measured using radio waves.

As a further alternative, the heart rate may be counted per unit time or may be measured as an amount of change based on regression curves.

In a case where the skin potential is measured as a physiological index, at least two electrodes are attached to appropriate positions of the body, and a difference in potential between these electrodes is taken to obtain measurements. The device for measuring the skin potential (referred to as the skin potential meter) may be a wearable device such as a smartwatch or may be included in a VR headset.

The skin potential may be measured as a value at the point in time of measurement, as an average of measured values per unit time, or as an amount of change based on regression curves.

The heart rate meter and the skin potential meter may be configured to be included in a single device, such as in the smartwatch or the VR headset. Also, the method of measuring the skin potential is not limited to the above-described method and may involve using the line of sight and body movement of the user in measuring the heart rate and the skin potential.

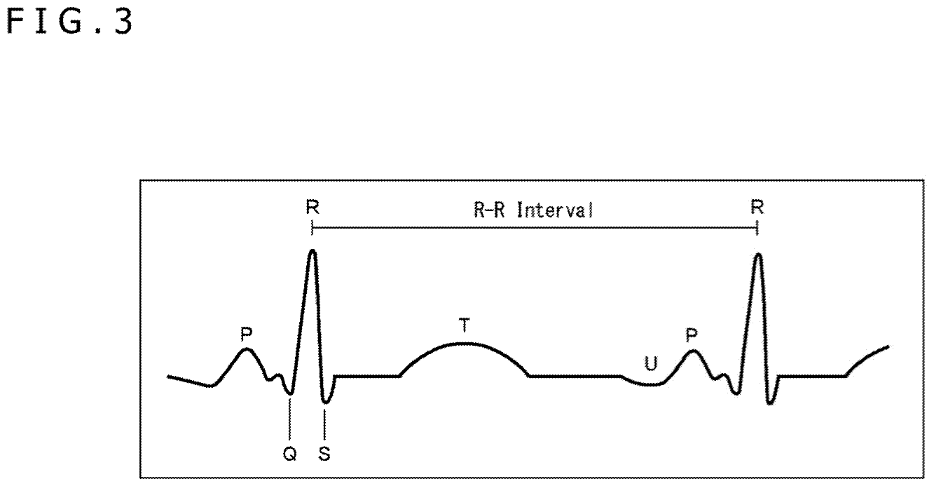

Also, a method has been proposed of measuring the sympathetic and parasympathetic nervous systems by keeping track of the heart rate. FIG. 3 depicts a waveform of the general heart rate curve. In FIG. 3, the horizontal axis represents timeline (sample axis), and the vertical axis denotes potential. As indicated in FIG. 3, the general heart rate curve presents a P wave, a Q wave, an R wave, an S wave, a T wave, and a U wave, in that order.

The time interval of the R wave is referred to as RRI (RR Interval) and is handled as heart rate interval data. Variations of the RRI are used as an index for analyzing the heart rate data. Given RRI time-series data, the following indexes may be used to quantify variations in the sympathetic and parasympathetic nervous systems.

### Frequency Domain Index

Low frequency power (LF): power at 0.05 to 0.15 Hz (low frequency band)

### Reflection of Activity of Sympathetic Nervous System

High frequency power (HF): power at 0.15 to 0.40 Hz (high frequency band)

### Reflection of Activity of Parasympathetic Nervous System

LF/HF ratio: ratio between LF and HF

The HF and LF of heart rate variations are known to vary depending on the balance between the sympathetic and parasympathetic nervous systems in a state of tension. Also, stress represents a balance between the sympathetic and parasympathetic nervous systems under tension. When the sympathetic nervous system is in a state of tension, the test subject may be defined to be in a stressed state; when the parasympathetic nervous system in is a state of tension, the test subject may be defined to be in a relaxed state.

That is, the relation between the heart rate and the stress is known to be such that, when a stress is experienced, the sympathetic nervous system is activated, and the parasympathetic nervous system is reduced in activity. In a case where the value of the LF/HF ratio is high, a stress is estimated to be experienced.

In a relaxed state, the HF component is relatively large, so that the LF/HF ratio becomes small. In a stressed state, by contrast, the LF component is large relative to the HF component, so that the LF/HF ratio becomes large. By using such specificity, for example, it is possible to determine that a stressed state is in effect if the LF/HF ratio is equal to or higher than a predetermined threshold value. However, a specific LF/HF ratio as the criterion above which a stressed state is recognized varies depending on the individual differences and on the conditions of measurement. In view of this, a reference value may be measured and determined beforehand for each individual subject to measurement, and the reference value thus determined may be used in setting the threshold value.

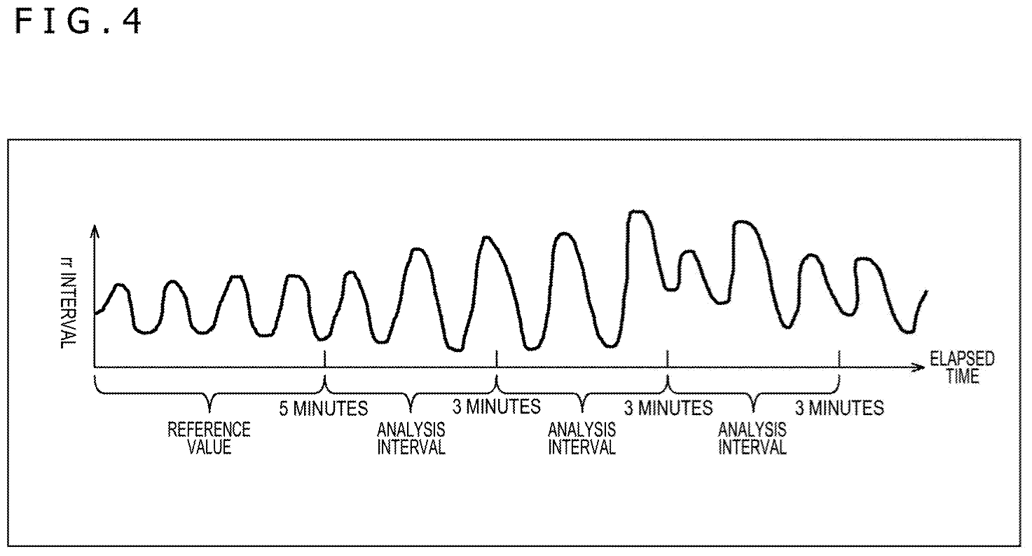

In a case where the above method is used to measure the sympathetic and parasympathetic nervous systems, it takes a period of time to acquire the HF component, LF component, and reference value. Therefore, in actual measurement, as indicated in FIG. 4, the heart rate is first measured for five minutes in a normal state. Thereafter, an analysis interval is appropriated every three minutes. In each analysis interval, estimates are made to determine whether or not a stress is experienced. During the analysis intervals, the RRI data for extracting the LP/HF value is accumulated. The HF component and the LF component are extracted from the accumulated data.

Although the interval for calculating the reference value is indicated to be five minutes and the analysis interval to be three minutes in FIG. 4, these time intervals are only examples and are not limitative of the length of time for such purposes. Thus, the analysis may be performed in a shorter period of time.

### <Exemplary System Configuration>

Explained below is an information processing apparatus that intervenes to reduce the stress estimated to be experienced by the user on the basis of measured physiological indexes.

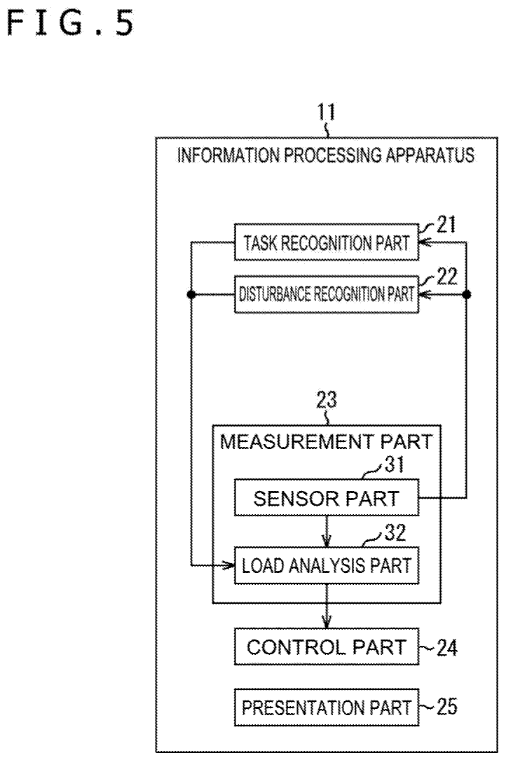

FIG. 5 is a diagram depicting an exemplary configuration of an information processing apparatus. An information processing apparatus 11 in FIG. 5 is configured to be incapable of controlling tasks or disturbances on its side.

The information processing apparatus 11 includes a task recognition part 21, a disturbance recognition part 22, a measurement part 23, a control part 24, and a presentation part 25. The measurement part 23 includes a sensor part 31 and a load analysis part 32.

The task recognition part 21 recognizes tasks. In a case where tasks cannot be directly recognized as when the information processing apparatus 11 itself offers no task, the task recognition part 21 estimates tasks using sensor information from the sensor part 31. The disturbance recognition part 22 recognizes disturbances. In a case where disturbances cannot be directly recognized, the disturbance recognition part 22 estimates disturbances also using the sensor information from the sensor part 31.

For example, in a case where the information processing apparatus 11 supports semi-autonomous driving, the task recognition part 21 recognizes as tasks such readings as driving at high speed and driving for an extended period of time, by acquiring information regarding vehicle velocity and travel time, for example. Also, the disturbance recognition part 22 recognizes as disturbances the external conditions of the vehicle, such as rainfall and appearance of fog.

Also, in a case where the information processing apparatus 11 supports remote control of a drone or a robot, for example, the tasks and disturbances may be recognized by monitoring the communication status and the amount of noise appearing on the display.

The sensor part 31 in the measurement part 23 includes a camera, a microphone, and contact-type sensors. The sensor part 31 includes sensors for measuring the user's physiological indexes and thus includes the sensors capable of measuring heart rate and skin potential as discussed above.

The sensor part 31 also includes, for example, a speedometer and an odometer of a vehicle and sensors for sensing disturbances such as rainfall conditions and temperature. Also, as discussed above, the task recognition part 21 may be configured to recognize tasks, and the disturbance recognition part 22 may be configured to recognize disturbances, on the basis of the data obtained by the sensor part 31.

The load analysis part 32 in the measurement part 23 estimates a workload and a visual load using the information regarding the tasks recognized by the task recognition part 21, information regarding the disturbances recognized by the disturbance recognition part 22, and diverse information obtained by the sensor part 31. Such estimates may be made through the use of the above-described methods.

The control part 24 provides the control that can be performed by the system, on the basis of the results of analysis by the load analysis part 32. In a case where the information processing apparatus 11 constitutes part of a system that supports semi-autonomous driving, the control part 24 controls the speed of the wipers and changes the currently presented information on the basis of the results of analysis by the load analysis part 32. The presentation part 25 presents the information as needed.

It is to be noted that the configuration of the information processing apparatus 11 in FIG. 5 is only an example and is not limitative of how the information processing apparatus 11 may be configured. Also, the sensor part 31, load analysis part 32, and control part 24 may be distributed in multiple devices when configured. The information processing apparatus 11 may also be configured in such a manner that some parts thereof, such as the load analysis part 32 and the control part 24, are located on servers.

### <Another Exemplary Configuration of the Information Processing Apparatus>

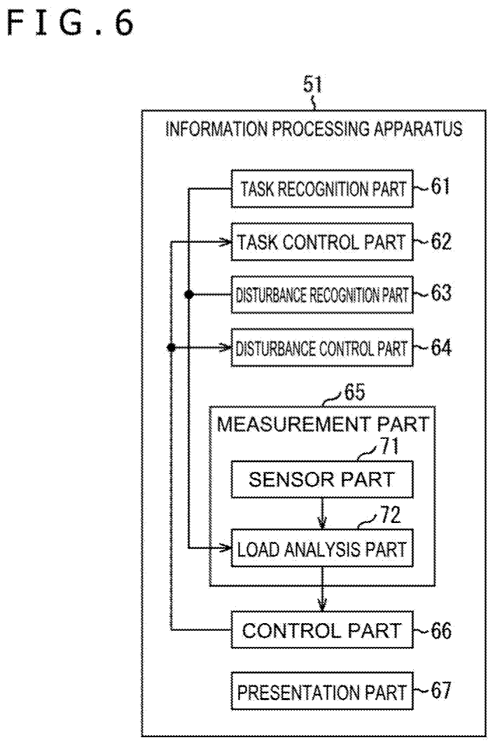

FIG. 6 is a diagram depicting another exemplary configuration of an information processing apparatus. An information processing apparatus 51 in FIG. 6 is configured to control at least one of the tasks and the disturbances on the apparatus side. For example, the information processing apparatus 51 may constitute part of an apparatus that offers games.

The information processing apparatus 51 includes a task recognition part 61, a task control part 62, a disturbance recognition part 63, a disturbance control part 64, a measurement part 65, a control part 66, and a presentation part 67. The measurement part 65 includes a sensor part 71 and a load analysis part 72.

The task recognition part 61 recognizes tasks. The task control part 62 controls the tasks. In a case where the information processing apparatus 51 is an apparatus that offers games, the tasks may involve defeating the enemy and solving mysteries during games, for example. Such tasks can be controlled by the task control part 62.

The task recognition part 61 can also recognize the task content being controlled by the task control part 62. The task recognition part 61 and the task control part 62 may be configured inside the same module.

The disturbance recognition part 63 recognizes disturbances. The disturbance control part 64 controls the disturbances. Also, in the case where the information processing apparatus 51 is an apparatus that offers games, the disturbance recognition part 63 recognizes the delay caused by a decreasing communication rate over the network or the noise appearing on the screen as the disturbances.

Also, in the case of the delay attributable to the decreasing communication rate, the disturbance control part 64 performs control to conduct communication in a narrower bandwidth, thereby suppressing the disturbance. If the amount of the noise generated on the screen is increased as a result of suppression of the disturbance, the increased nose amount is recognized by the disturbance recognition part 63. The disturbance recognition part 63 and the disturbance control part 64 may be configured inside the same module.

As with the measurement part 23 of the information processing apparatus 11 in FIG. 5, the measurement part 65 acquires diverse information via the sensor part 71. By use of the information thus acquired, of the information regarding the tasks recognized by the task recognition part 61, and of the information regarding the disturbances recognized by the disturbance recognition part 63, the load analysis part 72 analyzes the load experienced by the user.

On the basis of the result of analysis by the load analysis part 72, the control part 66 performs control to reduce the load on the user. For example, the control part 66 may instruct the task control part 62 to lower the difficulty level of the tasks. As another example, the control part 66 may instruct the disturbance control part 64 to secure a communication band on the network in conducting communication.

The presentation part 67 presents the information to the user as needed.

It is to be noted that the configuration of the information processing apparatus 51 in FIG. 6 is only an example and is not limitative of how the information processing apparatus 51 may be configured. Also, the sensor part 71, load analysis part 72, and control part 66 may be distributed in multiple devices when configured. Also, the information processing apparatus 51 may also be configured in such a manner that some parts thereof, such as the load analysis part 72 and control part 66, are located on servers.

Also, preferably, a system may be constructed to include both the information processing apparatus 11 and the information processing apparatus 51, one of the two being assigned control to perform processing under predetermined conditions. Alternatively, each of the apparatuses may carry out its own processing. Fr example, a system may be created to permit switching between the two apparatuses. For example, in a case where the tasks are given by the system side, the information processing apparatus 51 may perform the processing; in a case where the tasks are not controlled by the system side, the information processing apparatus 11 may carry out the processing.

### <First Process Regarding Interventions>

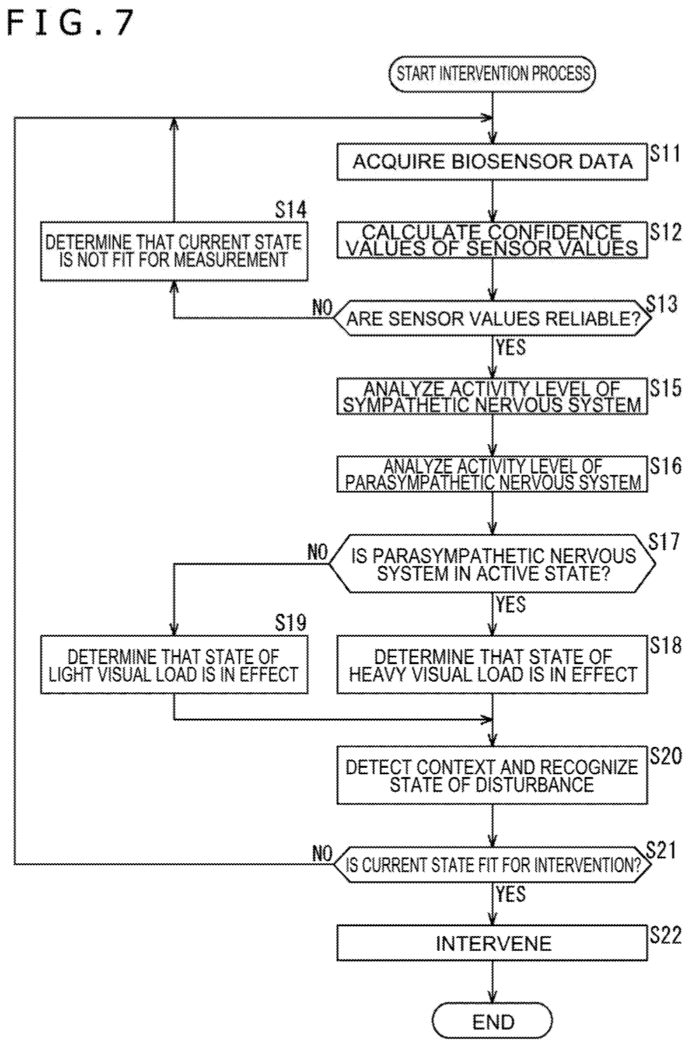

Explained below with reference to the flowchart in FIG. 7 is a process performed by either the information processing apparatus 11 or the information processing apparatus 51. Although the explanation below uses an example in which the information processing apparatus 51 performs the processing, basic processes are carried out likewise by the information processing apparatus 11.

In step S11, biosensor data is acquired. The biosensors included in the sensor part 71 measure the heart rate and skin potential of the user. For example, a sensor attached to the steering wheel of the vehicle measures the skin potential, and a camera installed in the vehicle interior captures images that are analyzed to measure the heart rate.

The diverse methods discussed above may be used to acquire the biosensor data. Also, the above-described methods may be supplemented with other arrangements such as measuring the pupil size and brain waves, the result of the measurement being used in conjunction with camera feed to recognize the status of the user. The result of such recognition may be utilized in combination with the data from the biosensors.

In step S12, the degree of reliability of the sensor values is calculated. The biosensor data acquired in step S11 is used here. In later steps, the activity level of the sympathetic nervous system and that of the parasympathetic nervous system are analyzed. What is calculated in step S12 is an indicator indicating whether or not the biosensor data acquired in step S11 can be used.

For example, in a case where the user is moving, the user's heart rate tends to be disturbed. It follows that the heart rate data acquired while the user is moving has a low degree of reliability. In view of this and by analyzing the images captured of the user by the camera installed in the vehicle interior, it is possible to determine whether or not the user is in motion. Also, in a case where a pressure sensor is attached to the vehicle seats, it is possible to determine whether or not the user is moving by use of the data from the pressure sensor.

The data obtained from the biosensors in the case where it is determined that the user is moving is assumed to have a low degree of reliability and is thus not utilized. On the other hand, in a case where it is determined that the user is not moving, i.e., in a case where the user is determined to be at rest, the data from the biosensors is assumed to have a high degree of reliability and is thus used in later steps. Such degrees of reliability are calculated in step S12.

In step S13, the load analysis part 72 determines whether or not the sensor values are reliable on the basis of the reliability value calculated in step S12. In a case where it is determined in step S13 that the sensor values are not reliable, control is transferred to step S14. In step S14, it is determined that the current state is not fit for measurement. Control is then returned to step S11, and the subsequent steps are repeated.

On the other hand, in a case where it is determined in step S13 that the sensor values are reliable, control is transferred to step S15.

In step S15, the load analysis part 72 analyzes the activity level of the sympathetic nervous system. The activity level of the sympathetic nervous system is analyzed by use of skin potential measurements. The skin potential responds to the activity of the sympathetic nervous system and is changed due to the presence of a mental stress or as a result of a close call, for example. In step S15, the measurements of the skin potential are used for analysis. In a case where the measured values are raised above the reference value, the activity level of the sympathetic nervous system is determined to be high.

In step S16, the activity level of the parasympathetic nervous system is analyzed. The measurements of the heart rate are used to analyze the activity level of the parasympathetic nervous system. The heart rate changes depending on the balance between the sympathetic nervous system and the parasympathetic nervous system. In a case where the heart rate is reduced, it is determined that the activity level of the parasympathetic nervous system is high.

Also, the activity level of the parasympathetic nervous system is determined by the results of analyzing the activity level of the sympathetic nervous system and that of the parasympathetic nervous system. For example, in a case where the heart rate is reduced relative to the reference value or where the heart rate is lower than a threshold value despite the activity level of the sympathetic nervous system being high, it is determined that the parasympathetic nervous system is in an active state.

Also, in the case where the parasympathetic nervous system is in an active state, the visual load can be determined to be heavy. On the other hand, in the case where the parasympathetic nervous system is not in an active state, the visual load can be determined to be light.

In step S17, it is determined whether or not the parasympathetic nervous system is in an active state through the use of the analysis results from step S16. In a case where it is determined in step S17 that the parasympathetic nervous system is in an active state, control is transferred to step S18. In step S18, the visual load is determined to be heavy. Control is then transferred to step S20.

On the other hand, in a case where it is determined in step S17 that the parasympathetic nervous system is not in an active state, control is transferred to step S19. In step S19, the visual load is determined to be light. Control is then transferred to step S20.

In step S20, the current context is detected, and the state of disturbance is recognized. The processing in step S20 involves determining whether or not to intervene to reduce the stress of the user and, in the event of deciding on an intervention, determining what kind of intervention can be made. Since the determination and handling of the intervention varies depending on the circumstances, a specific occurrence with which the user is currently experiencing the load is first determined, and suitable processes are then established to intervene to reduce the load.

In a case where control is transferred from step S18 to step S20, i.e., in a case where the visual load is determined to be heavy, an intervention may be made, for example, to announce an incoming email not on the spot but later when the load is determined to be reduced.

As another example, in a case where the visual load is determined to be heavy and a heavy rainfall is recognized as a contributing disturbance, an intervention may be made to increase the wiper speed so as to clear the vision. In a case where the inter-vehicle distance is recognized to be narrow, for example, an intervention may be made to increase the inter-vehicle distance.

On the other hand, in a case where control is transferred from step S19 to step S20, i.e., in a case where the visual load is determined to be light, an intervention may be made, for example, to announce an incoming email on the spot (the currently executing process may be maintained if so desired).

The above-described cases of interventions are only examples. These and other interventions not discussed above may be performed as needed.

In step S21, it is determined whether or not the current state is fit for intervention. It is determined here whether or not the intervention set in step S20 can actually be made in the current state. For example, in a case where an intervention is set to increase the wiper speed but where the wiper speed is already at maximum, it is determined that the current state is not fit for intervention.

In a case where it is determined in step S21 that the current state is not fit for intervention, control is returned to step S11, and the subsequent steps are repeated. On the other hand, in a case where it is determined in step S21 that the current state is fit for intervention, control is transferred to step S22, and the intervention is carried out.

In such a manner, the physiological indexes of the user are measured, and the results of the measurement are used to determine whether or not the user is experiencing a load. In the case where it is determined that the user is experiencing the load, an intervention is performed to reduce the load.

### <Second Process Regarding Interventions>

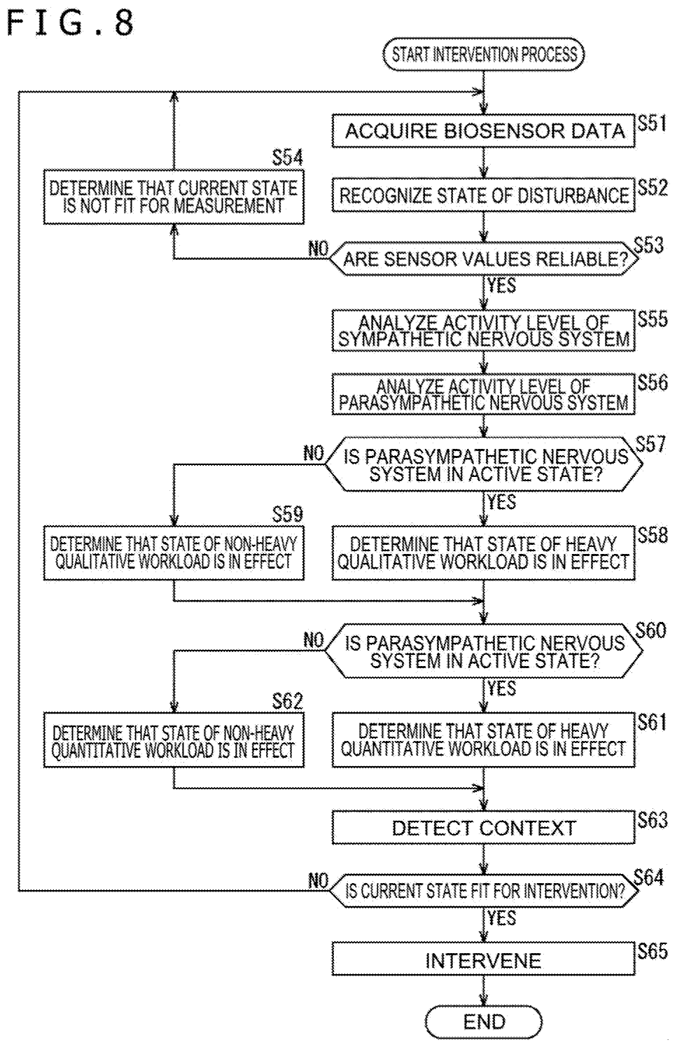

Explained below with reference to the flowchart in FIG. 8 is another process performed by either the information processing apparatus 11 or the information processing apparatus 51. The process regarding interventions, as explained below by referencing the flowchart in FIG. 8, represents a case in which the fatigued state of the user is detected and an intervention is made in a manner corresponding to the detected fatigued state.

In step S51, the biosensor data is acquired. The processing in step S51 may be performed in a manner similar to that in step S11 (FIG. 7).

In step S52, the state of disturbance is recognized. For example, a method of disturbance measurement may involve sensing the factors contributing to the disturbance in a case where the contributing factors such as a communication channel and noise are already known, in order to recognize the state of disturbance. Another method may involve sensing the status or the surroundings of the user so as to recognize the state of disturbance. For example, the temperature and rainfall conditions are recognized.

The recognition of disturbance involves recognizing the information that comes from the sensor part 71 and is necessary for determining whether or not the current state is fit for correctly recognizing the activity state of the sympathetic nervous system and that of the parasympathetic nervous system. The processing in step S52 corresponds to that in step S12 (FIG. 7) and may be performed in a manner similar to step S12.

Also, the disturbances are attributable to an environmental factor and a sensor factor. A typical disturbance attributable to the environmental factor may be a close call (e.g., rush-out of a person or a vehicle) during driving. Such a close call (disturbance) may be estimated from the vehicle sensors and from the status of the user.

Changes in the physiological indexes caused by the disturbance such as a close call are deemed to be temporary. Thus, in the case of the disturbance such as the close call, the data acquired from the sensor part 71 may be determined not to be used.

As another example, starting and stopping during driving can be estimated from the status of the vehicle. At a start or a stop during driving, the heart rate of the user is likely to change. Thus, in a case where a started or stopped state is recognized as a disturbance by sensing of the vehicle status, the biosensor data (heart rate data) may be treated as that temporarily changed because of the disturbance. The data acquired from the sensor part 71 may then be determined not to be used.

A state where the user is being spoken to by another user during game play is also recognized as a disturbance. A state where a specific event takes place during games is also recognized as a disturbance. These disturbances can temporarily affect changes in the user's heart rate. Thus, in a case where such disturbances are recognized, the biosensor data (heart rate data) may be treated as having been temporarily changed by the influence of the disturbances. The data acquired from the sensor part 71 may then be determined not to be used.

A typical disturbance attributable to the sensor factor is a body movement of the user. The user's body movement can be measured from data coming from the camera or from the pressure sensor attached to the vehicle seats. In a case where the user, upon moving his or her body, has moved out of the imaging range of the camera, for example, accurate imaging cannot be performed, and the data acquired by analyzing the images captured in such a state is considered to be low in reliability.

Also, as explained above in connection with step S12 (FIG. 7), in the case where the user is moving, the heart rate is likely to change. The data acquired from the sensor part 71 in such a state is considered to be low in reliability and may be determined not to be used.

Another typical disturbance attributable to the sensor factor is an obstruction of the field of view of the camera. In a case where an object crossing in front of the camera prevents the camera from capturing images of the user or the external environment, for example, the data from the camera (sensor part 71) is considered to be low in reliability and may be determined not to be used.

In step S52, the state of disturbance is recognized by the disturbance recognition part 63. In step S53, the load analysis part 72 determines whether or not the sensor values are reliable. As discussed above, there are cases where the sensor values are not reliable depending on the status of disturbance. In a case where it is determined in step S53 that the sensor values are not reliable, control is transferred to step S54. In step S54, it is determined that the current state is not fit for measurement. Control is then returned to step S51, and the subsequent steps are repeated.

On the other hand, in a case where it is determined in step S53 that the sensor values are reliable, control is transferred to step S55. In step S55, the activity level of the sympathetic nervous system is analyzed. Also, in step S56, the activity level of the parasympathetic nervous system is analyzed. The processing in steps S55 and S56 may be carried out in a manner similar to the processing in steps S15 and S16 (FIG. 7).

In step S57, it is determined whether or not the sympathetic nervous system is in an active state. In a case where the measured values of skin potential are raised above the reference value, the analyzed activity level of the sympathetic nervous system is determined to be high. In a case where the measured values of skin potential are changed, the sympathetic nervous system is determined to be in a highly active state. In that case, it is determined that the user is in a state of heavy workload, particularly of heavy quantitative load.

In a case where it is determined in step S57 that the sympathetic nervous system is in the active state, control is transferred to step S58. In step S58, it is determined that a state of heavy qualitative workload is in effect. Control is then transferred to step S60.

On the other hand, in a case where it is determined in step S57 that the sympathetic nervous system is not in the active state, control is transferred to step S59. In step S59, it is determined that a state of light qualitative workload is in effect. Control is then transferred to step S60.

In step S60, it is determined whether or not the parasympathetic nervous system is in an active state. As explained above with reference to FIG. 2, in the case where the measured values of the parasympathetic nervous system are raised above the reference value, it is determined that a state of heavy quantitative workload is in effect. Also, in a case where the parasympathetic nervous system is determined to be in the active state on the basis of the results of analysis of the sympathetic and parasympathetic nervous systems, it is determined that a state of heavy visual load (state of concentration) is in effect.

In a case where it is determined in step S60 that the parasympathetic nervous system is in the active state, control is transferred to step S61. In step S61, it is determined that a state of heavy quantitative workload is in effect. Control is then transferred to step S63.

On the other hand, in a case where it is determined in step S60 that the parasympathetic nervous system is not in the active state, control is transferred to step 62. In step S62, it is determined that a state of non-heavy quantitative workload is in effect. Control is then transferred to step S63.

In step S63, the current context is determined. The processing in step S63, as in the case of the processing in step S20 (FIG. 7), involves determining whether or not to intervene to reduce the stress of the user and, in the event of deciding on an intervention, determining what kind of intervention can be made.

Carrying out the processing in steps S57 through S61 determines whether the user is experiencing a qualitative workload or a quantitative workload. In a case where it is determined that the qualitative workload is experienced, an intervention is determined to be made to reduce the qualitative component of the workload, such as by lowering the difficulty level of work.

Also, in a case where it is determined that the quantitative workload is experienced, an intervention is determined to be made to reduce the quantitative component of the workload, such as by prompting the user to take a rest (i.e., to interrupt work).

Also, in a case where it is determined that both the quantitative workload and the qualitative workload are light, an intervention is determined to be made to increase the quantitative and qualitative components of the workload, such as by raising the difficulty level of work.

When the specific content of intervention is determined in step S63, step S64 is reached, and it is determined whether or not the current state is fit for intervention. If the current state is determined to be fit for intervention, step S65 is reached, and the intervention is performed. On the other hand, in a case where the current state is determined not to be fit for intervention, control is returned to step S51, and the subsequent steps are repeated.

As described above, the physiological indexes of the user are measured, and the results of the measurement are used to determine whether or not the user is experiencing a load. In the case where it is determined that the user is experiencing a load, an intervention is made to reduce the load. Because it is possible to determine the type of the load experienced by the user, i.e., whether the user is experiencing a qualitative workload or a quantitative workload, more appropriate interventions can be performed.

### Application Examples

Some application examples of the information processing apparatus performing the above-described processes are explained below. As discussed above, the information processing apparatus may be used remotely to operate a drone. Explained here is an exemplary case in which the information processing apparatus is used to monitor a bridge using a drone.

The user wears suitable equipment such as a VR headset. It is noted that the VR headset is not mandatory and may be replaced by a smart glass offering AR. The equipment need not be attached to a body portion of the user. Alternatively, the user may sit in front of a display to watch the screen.

The user also wears the sensors for measuring the heart rate and the skin potential. The sensors may be included in the headset or may be configured as a band to be attached to an appropriate body portion of the user such as the arm or the foot. As another alternative, a camera may be used to capture and analyze images for sensing purposes.

When work is started, the drone is made to fly over inspection points to check for abnormalities. For example, the user (i.e., worker) may only operate the drone and let the drone side check for any abnormality.

As another example, the drone may be allowed to fly autonomously on the basis of a flight plan and to check concurrently for abnormalities. The worker monitors the state of the drone in autonomous flight as well as the status of abnormality detection. In the event of an irregular situation, the worker performs processes (control) to deal with the irregular situation.

Also, in a case where the drone detects an abnormality, the drone flags the point in time of abnormality detection. The worker monitors whether the detection is not erroneous and, if the detection turns out to be erroneous, carries out processes to correct the error. Also, in a case where the worker detects an abnormality, the worker performs processes to flag the situation in which the abnormality is detected.

Such processes are repeated while the bridge is being monitored by use of the drone.

During monitoring of the bridge, the information processing apparatus using the above-described technology monitors the load state of the user. In a case where it can be determined that the user is experiencing a load, the information processing apparatus can intervene to reduce the load.

For example, the workload and the visual load of the worker are separately estimated. The visual load involves a load due to a drop in the quality of images being watched by the worker, or a load attributable to an impediment to the work of paying visual attention.

In another example, the occurrences of events incur a load. The load attributable to the occurrences of events involves the start, stop, and success of a task. The occurrences of these events can be detected on the side of the information processing apparatus and thus can be controlled through cancellation, for example.

The body movement and gestures of the user are detected by use of an acceleration sensor and a camera. Control is performed in such a manner that the values measured while the user is moving (in motion) are not used for evaluation.

Also, the level of skill varies for each worker. The manner of experiencing the load also varies for each worker. The differences between individuals are controlled to be cancelled in line with their skill levels on normal tasks and by calibration based on practice tasks. Preferably, the information regarding the user at rest may be acquired beforehand and used as the reference with which comparisons are made to estimate the load experienced by the user.

The present technology may also be applied to the field of medicine. For example, the technology may be applied to telesurgery, to surgery by a surgeon wearing an HMD (Head Mounted Display), and to surgery through viewing of monitor screens.

The present technology may also be applied to the navigation of mobility devices such as vehicles, ships, and aircraft during manual driving or operation, or to the control of vehicles during automated driving, semi-autonomous driving, or driving by ADAS (Advanced Driver Assistance System).

The present technology may also be applied to the remote control of robots. For example, the technology may be applied to remote work using a drone or a mobile robot, as well to remotely controlled operations using tele-existence technology.

The present technology may also be applied to telepresence setups. For example, the technology may be applied to distance education, telemedicine, and remote consulting.

The present technology may also be applied to the offering of games.

### <Recording Medium>

The series of processes described above may be executed either by hardware or by software. In the case where these processes are to be carried out by software, the programs constituting the software are installed into a suitable computer. Such computers may include those with the software incorporated in their dedicated hardware beforehand, and those such as a general-purpose personal computer capable of executing diverse functions based on various programs installed therein.

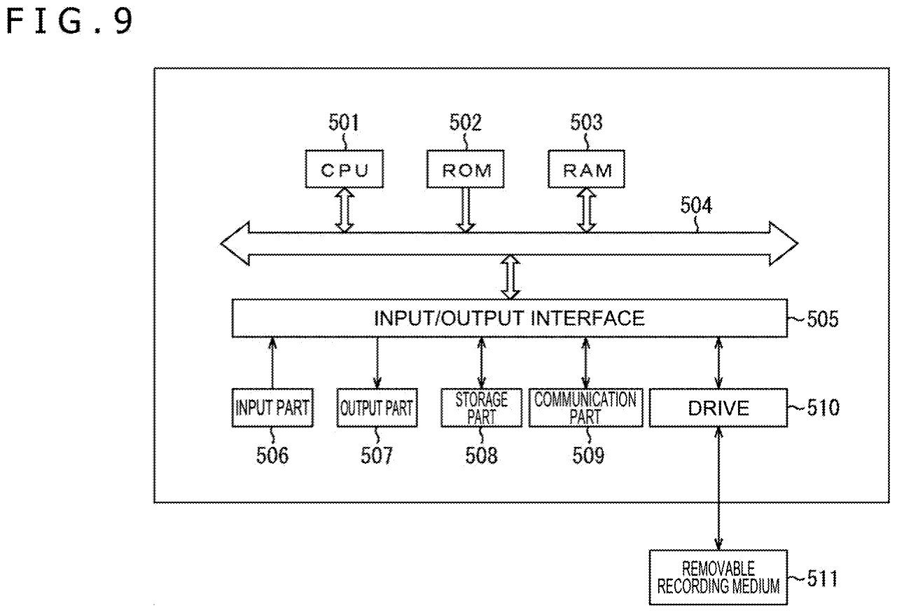

FIG. 9 is a block diagram depicting an exemplary hardware configuration of a computer executing the above-described processes using programs. In the computer, a CPU (Central Processing Unit) 501, a ROM (Read Only Memory) 502, and a RAM (Random Access Memory) 503 are interconnected via a bus 504. The bus 504 is further connected with an input/output interface 505. The input/output interface 505 is connected with an input part 506, an output part 507, a storage part 508, a communication part 509, and a drive 510.

The input part 506 includes a keyboard, a mouse, and a microphone. The output part 507 includes a display and speakers. The storage part 508 includes a hard disk and a nonvolatile memory. The communication part 509 includes a network interface. The drive 510 drives a removable medium 511 such as a magnetic disk, an optical disk, a magneto-optical disk, or a semiconductor memory.

In the computer configured as described above, the CPU 501 performs the above-mentioned series of processing by loading appropriate programs from the storage part 508 into the RAM 503 via the input/output interface 505 and the bus 504, for example, and by executing the loaded programs.

The programs to be executed by the computer (CPU 501) may be recorded, for example, on the removable medium 511 as a packaged medium when offered. The programs may also be offered via a wired or wireless transmission medium such as local area networks, the Internet, and digital satellite broadcasting.

In the computer, the programs may be installed into the storage part 508 from the removable medium 511 attached to the drive 510 via the input/output interface 505. The programs may also be installed into the storage part 508 after being received by the communication part 509 via a wired or wireless transmission medium. The programs may alternatively be preinstalled in the ROM 502 or in the storage part 508.

It is noted that each of the programs to be executed by the computer may be carried out chronologically in the depicted sequence of this specification, in parallel with each other, or in otherwise appropriately timed fashion such as when the program is invoked as needed.

Also, in this description, the term “system” refers to a whole apparatus including multiple devices.

It is noted that the advantageous effects stated in this description are only examples and not limitative of the present disclosure that may provide other advantages as well.

It is noted that the present technology is not limited to the preferred embodiment discussed above and can be implemented in diverse variations so far as they are within the scope of this technology.

It is noted that the present technology can also take the following configurations.

(1)

An information processing apparatus including:
an estimation part configured to estimate a load experienced by a user, on the basis of data from a sensor measuring a physiological index of the user, in which
the estimation part estimates, as the load, at least one of a workload experienced by the user carrying out a predetermined task and a visual load experienced by the user making alert concentration.
   (2)

The information processing apparatus according to (1) above, in which
the workload includes a qualitative workload experienced by carrying out the task per unit time and a quantitative workload accumulated by continuously carrying out the task, and
the estimation part estimates, as the load, at least one of the qualitative workload, the quantitative workload, and the visual load.
   (3)

The information processing apparatus according to (1) or (2) above, in which
the physiological index includes a heart rate, and
the estimation part estimates a state in which the visual load is experienced in response to a change in the heart rate.
   (4)

The information processing apparatus according to (2) or (3) above, in which
the physiological index includes a heart rate and skin potential,
in a case where there is a drop in the heart rate but no change in the skin potential, the estimation part estimates a state in which the visual load is experienced,
in a case where there is an increase in the heart rate but no change in the skin potential, the estimation part estimates a state in which the quantitative workload is experienced, and
in a case where there is an increase in the heart rate as well as an increase in the skin potential, the estimation part estimates a state in which the qualitative workload is experienced.
   (5)

The information processing apparatus according to any one of (1) to (4) above, in which
the physiological index includes a heart rate, and
the estimation part determines that a parasympathetic nervous system is in an active state in response to a change in the heart rate and estimates a state in which the visual load is experienced.
   (6)

The information processing apparatus according to any one of (1) to (5) above, in which
the physiological index includes skin potential, and
the estimation part determines that a sympathetic nervous system is in an active state in response to a change in the skin potential and estimates a state in which the workload is experienced.
   (7)

The information processing apparatus according to any one of (1) to (6) above, in which
the physiological index includes a heart rate and skin potential, and
the estimation part determines that a parasympathetic nervous system is in an active state in response to a change in the heart rate, determines that a sympathetic nervous system is in an active state according to the skin potential, and estimates whether or not the load is experienced by the user on the basis of the active state of the parasympathetic nervous system and the active state of the sympathetic nervous system.
   (8)

The information processing apparatus according to any one of (1) to (7) above, in which
the physiological index includes a heart rate, and
the estimation part analyzes a high-frequency component and a low-frequency component of a change in the heart rate and estimates whether or not the load is experienced by the user on the basis of a ratio between the high-frequency component and the low-frequency component.
   (9)

The information processing apparatus according to (8) above, in which the load is estimated to be experienced by the user in a case where the ratio between the high-frequency component and the low-frequency component is higher than a reference value.

(10)

The information processing apparatus according to any one of (1) to (9) above, further including:
a control part configured to control an intervention to reduce the workload in a case where the workload is estimated to be experienced by the user and to control an intervention to reduce the visual load in a case where the visual load is estimated to be experienced by the user.
   (11)

The information processing apparatus according to any one of (2) to (10) above, further including:
a control part configured to control an intervention to reduce the qualitative workload in a case where the qualitative workload is estimated to be experienced by the user, to control an intervention to reduce the quantitative workload in a case where the quantitative workload is estimated to be experienced by the user, and to control an intervention to reduce the visual load in a case where the visual load is estimated to be experienced by the user.
   (12)

The information processing apparatus according to any one of (1) to (11) above, in which the estimation part determines whether or not the user is in a stationary state and does not perform the estimation in a case where the user is determined not to be in the stationary state.

(13)

The information processing apparatus according to any one of (1) to (12) above, in which the estimation part does not perform the estimation in a case where a disturbance is determined to have occurred.

(14)

An information processing method including:
causing an information processing apparatus to estimate a load experienced by a user on the basis of data from a sensor measuring a physiological index of the user; and
causing the information processing apparatus to estimate, as the load, at least one of a workload experienced by the user carrying out a predetermined task and a visual load experienced by the user making alert concentration.
   (15)

A program for causing a computer controlling an information processing apparatus to execute a process including:
estimating a load experienced by a user on the basis of data from a sensor measuring a physiological index of the user; and
estimating, as the load, at least one of a workload experienced by the user carrying out a predetermined task and a visual load experienced by the user making alert concentration.

### REFERENCE SIGNS LIST

11: Information processing apparatus
21: Task recognition part
22: Disturbance recognition part
23: Measurement part
24: Control part
25: Presentation part
31: Sensor part
32: Load analysis part
51: Information processing apparatus
61: Task recognition part
62: Task control part
63: Disturbance recognition part
64: Disturbance control part
65: Measurement part
66: Control part
67: Presentation part
71: Sensor part
72: Load analysis part

1. An information processing apparatus comprising:
an estimation part configured to estimate a load experienced by a user, on a basis of data from a sensor measuring a physiological index of the user, wherein
the estimation part estimates, as the load, at least one of a workload experienced by the user carrying out a predetermined task and a visual load experienced by the user making alert concentration.

2. The information processing apparatus according to claim 1, wherein
the workload includes a qualitative workload experienced by carrying out the task per unit time and a quantitative workload accumulated by continuously carrying out the task, and
the estimation part estimates, as the load, at least one of the qualitative workload, the quantitative workload, and the visual load.

3. The information processing apparatus according to claim 1, wherein
the physiological index includes a heart rate, and
the estimation part estimates a state in which the visual load is experienced in response to a change in the heart rate.

4. The information processing apparatus according to claim 2, wherein
the physiological index includes a heart rate and skin potential,
in a case where there is a drop in the heart rate but no change in the skin potential, the estimation part estimates a state in which the visual load is experienced,
in a case where there is an increase in the heart rate but no change in the skin potential, the estimation part estimates a state in which the quantitative workload is experienced, and
in a case where there is an increase in the heart rate as well as an increase in the skin potential, the estimation part estimates a state in which the qualitative workload is experienced.

5. The information processing apparatus according to claim 1, wherein
the physiological index includes a heart rate, and
the estimation part determines that a parasympathetic nervous system is in an active state in response to a change in the heart rate and estimates a state in which the visual load is experienced.

6. The information processing apparatus according to claim 1, wherein
the physiological index includes skin potential, and
the estimation part determines that a sympathetic nervous system is in an active state in response to a change in the skin potential and estimates a state in which the workload is experienced.

7. The information processing apparatus according to claim 1, wherein
the physiological index includes a heart rate and skin potential, and
the estimation part determines that a parasympathetic nervous system is in an active state in response to a change in the heart rate, determines that a sympathetic nervous system is in an active state according to the skin potential, and estimates whether or not the load is experienced by the user on a basis of the active state of the parasympathetic nervous system and the active state of the sympathetic nervous system.

8. The information processing apparatus according to claim 1, wherein
the physiological index includes a heart rate, and
the estimation part analyzes a high-frequency component and a low-frequency component of a change in the heart rate and estimates whether or not the load is experienced by the user on a basis of a ratio between the high-frequency component and the low-frequency component.

9. The information processing apparatus according to claim 8, wherein the load is estimated to be experienced by the user in a case where the ratio between the high-frequency component and the low-frequency component is higher than a reference value.

10. The information processing apparatus according to claim 1, further comprising:
a control part configured to control an intervention to reduce the workload in a case where the workload is estimated to be experienced by the user and to control an intervention to reduce the visual load in a case where the visual load is estimated to be experienced by the user.

11. The information processing apparatus according to claim 2, further comprising:
a control part configured to control an intervention to reduce the qualitative workload in a case where the qualitative workload is estimated to be experienced by the user, to control an intervention to reduce the quantitative workload in a case where the quantitative workload is estimated to be experienced by the user, and to control an intervention to reduce the visual load in a case where the visual load is estimated to be experienced by the user.

12. The information processing apparatus according to claim 1, wherein the estimation part determines whether or not the user is in a stationary state and does not perform the estimation in a case where the user is determined not to be in the stationary state.

13. The information processing apparatus according to claim 1, wherein the estimation part does not perform the estimation in a case where a disturbance is determined to have occurred.

14. An information processing method comprising:
causing an information processing apparatus to estimate a load experienced by a user on a basis of data from a sensor measuring a physiological index of the user; and
causing the information processing apparatus to estimate, as the load, at least one of a workload experienced by the user carrying out a predetermined task and a visual load experienced by the user making alert concentration.

15. A program for causing a computer controlling an information processing apparatus to execute a process comprising:
estimating a load experienced by a user on a basis of data from a sensor measuring a physiological index of the user; and
estimating, as the load, at least one of a workload experienced by the user carrying out a predetermined task and a visual load experienced by the user making alert concentration.
